(19) United States
(12) Patent Application Publication
(10) Pub. No.: US 2016/0121134 A1
(43) Pub. Date: May 5, 2016

(54) MEDICAL DEVICE FOR APPLYING NON-THERMAL PLASMA TO SELECTED TARGETS

(71) Applicant: EP Technologies LLC, Akron, OH (US)

(72) Inventors: Sameer Kalghatgi, Copley, OH (US); David Petrak, Mayfield Heights, OH (US); Tsung-Chan Tsai, Cuyahoga Falls, OH (US); Daphne Pappas Antonakas, Hudson, OH (US); Robert L. Gray, Hudson, OH (US)

(21) Appl. No.: 14/921,261

(22) Filed: Oct. 23, 2015

Related U.S. Application Data
(60) Provisional application No. 62/072,235, filed on Oct. 29, 2014.

(51) Int. Cl. A61N 1/44; A61B 18/04; H05H 1/24

(52) U.S. Cl. CPC A61N 1/44; H05H 1/2406; A61B 18/042; H05H 2001/2418

(57) ABSTRACT

A medical device for directly applying DBD plasma includes a plasma generation module having an electrode surrounded by a dielectric barrier. In one embodiment, when a high voltage source is applied to the electrode, plasma is produced from ambient gas on an outside surface of the plasma generation module. In another embodiment, an enclosure is deployed around the distal end of the plasma generation module surrounding the electrode. Gas flows past the electrode and into the enclosure. In some embodiments, some or all of the gas is removed or pumped out of the enclosure by suction/vacuum. When a high voltage source is applied to the electrode, plasma is produced using the gas flowing into the enclosure.

### RELATED APPLICATIONS

This application claims priority to and the benefits of U.S. Provisional Patent Application Ser. No. 62/072235 filed on Oct. 29, 2014 and entitled “MEDICAL DEVICE FOR APPLYING NON-THERMAL PLASMA TO SELECTED TARGETS,” which is incorporated herein by reference in its entirety.

### TECHNICAL FIELD

The present invention relates generally medical devices for applying non-thermal plasma, and more particularly, in some embodiments, to a medical device for directly applying dielectric barrier discharge (DBD) plasma to tissue inside a body cavity with minimal or no additional gas supply, and, in some embodiments, for applying non-thermal plasma in a microenvironment inside a body.

### BACKGROUND OF THE INVENTION

Recently, it has been discovered that non-thermal atmospheric pressure plasma may be useful for disinfection, wound treatment and other clinical applications. Prior art devices have used gas flow plasma generators, plasma jets and the like to generate plasma and to treat an internal area of the body. The gases these devices use and expel during plasma generation, such as argon and helium, may be harmful to the human body when left in a body cavity or if allowed to accumulate as they can decrease the levels of oxygen in organs and cause tissue damage.

### SUMMARY

Exemplary embodiments of medical devices and methods for non-thermal plasma application are disclosed herein. One exemplary embodiment of a medical device for direct DBD plasma application includes a proximal end and a distal end, a first channel extending between the two ends and having an opening at the distal end and a plasma generation module disposed in the first channel. The plasma generation module includes a distal end opposing a proximate end, an electrode located within the module at the distal end of the module and a dielectric barrier material surrounding the electrode. When a high voltage provided from a high voltage source is applied to the electrode, and the distal end of the module is brought in proximity to the tissue being treated, plasma is produced in ambient gas on an outside surface of the plasma generation module at the distal end of the module.

Another exemplary embodiment of a medical device for plasma application includes a proximal end and a distal end, a first channel extending between the two ends and having an opening at the distal end, and a plasma generation module disposed in the first channel. The plasma generation module includes a distal end opposing a proximate end, an electrode located within the module at the distal end of the module, a dielectric barrier material surrounding the electrode, a gas inlet at the proximal end of the module for receiving gas to flow through the module to a gas outlet at the distal end of the module, and a deployable enclosure at the distal end of the module wherein the deployable enclosure surrounds the electrode and gas outlet when deployed. When a high voltage source is applied to the electrode, plasma is produced using gas flowing near the electrode at the distal end of the plasma generation module.

An exemplary embodiment of a method for plasma application to an application surface includes deploying an enclosure to enclose a space at the end of the medical device and the application surface, flowing gas into the enclosure, removing gas from the enclosure to create slightly negative pressure in the enclosure and applying a high voltage from a high voltage source to an electrode to produce plasma in the gas flowing into the enclosure.

### BRIEF DESCRIPTION OF THE DRAWINGS

These and other features and advantages of the present invention will become better understood with regard to the following description and accompanying drawings in which:

FIG. 1 is a perspective view of an exemplary embodiment of a medical device for direct application of DBD plasma without a gas supply.

FIG. 2 is a perspective view of an exemplary embodiment of a medical device for direct application of DBD plasma with a gas flow and a flexible enclosure.

FIG. 3 is a perspective view of an exemplary embodiment of a plasma generation module with an undeployed flexible enclosure.

FIG. 4 is a schematic diagram of an exemplary embodiment of a plasma generation module with a deployed protective enclosure.

FIG. 5 is a schematic diagram of an exemplary embodiment of a plasma generation module without a gas flow and in operation.

FIG. 6 is a two-perspective view of an exemplary embodiment of a plasma generation module with a gas flow and a deployed protective flexible enclosure in operation.

### DETAILED DESCRIPTION

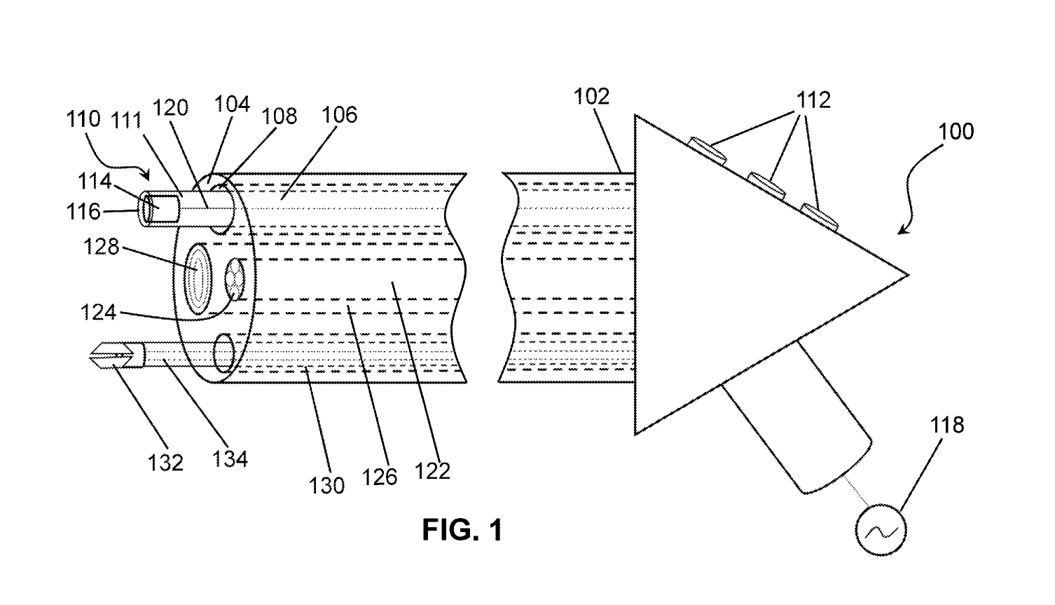

FIG. 1 is an exemplary embodiment of a medical device 100 for application of direct dielectric barrier discharge (DBD) plasma. The medical device 100 includes a proximal end 102 and a distal end 104. In some embodiments all or part of the medical device may be flexible, for example a flexible endoscope. In the case of a flexible endoscope, the flexible portion of the device may be made from any suitable flexible material such as polytetrafluoroethylene (PTFE), rubber, silicone or urethane. In another embodiment the medical device may be rigid, such as a typical laparoscope. A rigid medical device may be made from metal, such as, for example surgical stainless steel, or a rigid polymer or any other suitably rigid material. As disclosed herein, the medical device 100 and other embodiments may include any number of modules, features and devices. Those modules, features and devices may be made from the same, similar, or any other of the materials suitable for the body of the medical device itself.

A channel 106 extends between the two ends 102 and 104 and has an opening 108 at the distal end 104. A plasma generation module 110 is disposed in the channel 106. In some embodiments the plasma generation module 110 includes hollow tube 111 with its longitudinal axis disposed in the channel 106. The plasma generation module 110 fits within the channel 106 and has a smaller outside diameter than the inside diameter of the channel 106. The plasma generation module 110 may be movable within the channel 106 and may be extended out of the distal end 104 of the medical device 100 through the opening 108. In some embodiments the medical device 100 includes multiple plasma generation modules (not shown) each disposed in its own channel. In some embodiments, extension and retraction of the plasma generation module 110 may be controlled by a set of controls, such as controls 112 near the proximate end 102 of the medical device 100. The controls 112 may be mechanical, electrical, electronic, electromechanical or combinations thereof and may include knobs, buttons, switches and the like, or any combination thereof.

A high-voltage electrode 114 is located within the plasma generation module 110 at the distal end of the module. In some embodiments, the high voltage electrode 114 is made of a flat conductive material. In some embodiments, the high voltage electrode 114 contains copper, silver, brass, bronze, aluminum, stainless steel, gold, carbon nanotubes, carbon nanowires or the like, or mixtures of one or more of these conductive materials.

A dielectric barrier 116 at least partially surrounds the electrode 114. In some embodiments, the plasma generation module 110 is closed at its distal end so as to surround the electrode 114 and the material of the module 110 itself serves as the dielectric barrier. In some embodiments, the plasma generation module 110 may or may not be closed at its distal end and insulation around the high-voltage electrode 114 is the dielectric barrier material. Typical dielectric materials include, but are not limited to, glass, quartz, ceramics and polymers.

The high-voltage electrode 114 is connected, or connectable to a high-voltage source 118, for example by conductive wire 120. High-voltage source 118 may be a nanosecond pulsed power source, a microsecond pulsed power source, a picosecond pulsed power source, a sinusoidal power source, RF driven power source, pulsed DC or pulsed AC driven power source or the like. In addition, in some embodiments, the high-voltage source 118 includes one or more batteries (not shown) and circuitry necessary to convert the low voltage to a high-voltage AC source, or to a high voltage DC source. Amplitude of applied voltage may range from 1 kV to 30 kV, frequency of the sinusoidal voltage waveforms from 2 Hz to 1 MHz, repetition rate of the pulsed waveform from 2 Hz to 30 kHz, pulse duration from picosecond to millisecond and a duty cycle from 1%-100%. The plasma exposure can be applied in a continuous mode or through the application of one or more electrical pulses that are capable to generate the dielectric barrier discharges.

When a high-voltage provided from a high-voltage source is applied to the electrode 114, plasma is produced by the plasma generation module 110 at the distal end of the module. In these embodiments the object to which the plasma is being applied, for example bodily tissue, is grounded and serves as a second electrode to complete the circuit. In some embodiments, the second electrode is a floating ground. When high voltage is applied to the electrode 114, plasma forms in the atmospheric-pressure gas between the dielectric barrier 116 and the tissue. Thus, in these embodiments, there is no need for a dedicated external gas supply to generate plasma, and no foreign gasses are introduced into the body during plasma generation.

In some embodiments, a second channel 122 extends between the proximal and distal ends of the medical device 100. At least one light source 124 is disposed in the second channel 122. In some embodiments the light source 124 includes one or more light-emitting diodes (LEDs). The light source 124 may be fixed at or near the distal end of the channel 122 or may be recessed within the channel 122. In some embodiments the light source 124 is extendable out from the distal end of the channel 122. The light source 124 is connected, or connectable to a voltage source. The voltage source may be the same voltage source 118 connected to the high-voltage electrode 114 and may include circuitry necessary to convert the high voltage to a low-voltage AC or DC source. The light source may be controlled by a set of controls on the medical device 100 such as controls 112. In some embodiments, light source 124 includes fiber optics and the light source is located in the proximal end 102.

In some embodiments a third channel 126 extends between the proximal and distal ends of the medical device 100. A camera 128 is disposed in the third channel 126 for viewing from the distal end of the device 100. In some embodiments, the camera 128 may be a charge-coupled device, active-pixel sensor, or the like. The camera 128 may include one or more lenses and/or one or more lenses may be fixed between the camera 128 and the distal end of the channel 126. In some embodiments the camera 128 is extendable out from the distal end of the channel 126. The camera 128 is connected, or connectable to a voltage source. The voltage source may be the same voltage source 118 connected to the high voltage electrode 114 and may include circuitry necessary to convert the high voltage to a lower AC or DC source. In some embodiments the camera 128 is connected, or connectable to a computer or processing device that may also serve as a voltage source. In some embodiments the camera 128 is connected to a display device such as liquid-crystal display. The camera may be controlled from a connected computer or processing device, or may be controlled by controls on the medical device 100 such as controls 112.

In some embodiments a fourth channel 130 extends between the proximal and distal ends of the medical device 100. A pair of forceps 132 is disposed in the fourth channel 130 and is extendable out of the channel 130 at the distal end of the medical device 100. The forceps 132 may be used to grasp and/or cut material such as bodily tissue. The forceps 132 may be made of any suitable material, for example surgical stainless steel. In some embodiments the forceps 132 have a cup-shape to hold collected tissue when the forceps 132 are closed.

In some embodiments, retraction and extension of the forceps 132 are controlled by one or more operational wires 134. The wires in turn may be connected to a set of operational controls, such as controls 112. In some embodiments the operational wires 134 may also control the opening and closing of the forceps 132.

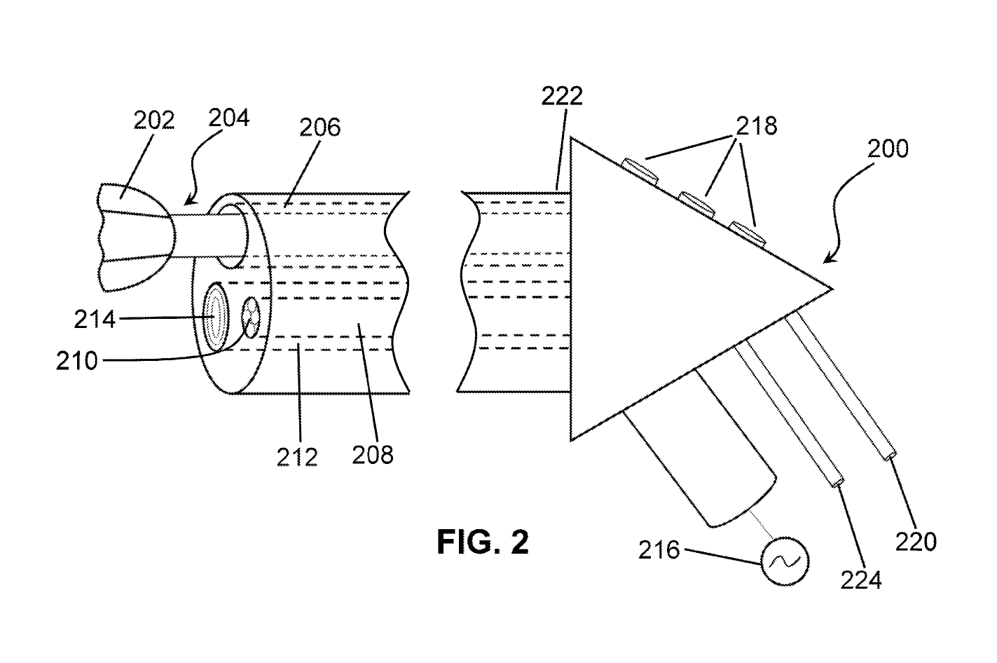

FIG. 2 illustrates an embodiment where medical device 200 uses a gas flow for plasma generation, but includes a deployable enclosure 202 for creating an enclosed microenvironment around the plasma generation module 204 to prevent gases from escaping into the body. As will be described in further detail, the enclosure 202 is deployable from a channel 206 of the medical device 200. The medical device 200 may include any number of additional channels housing other modules, features or devices, for example, channel 208 housing light source 210 or channel 212 housing camera 214. The modules, features or devices may be powered by power source 216, which may be any suitable power source, with or without additional circuitry, as described for previous embodiments. Deployment of the enclosure 202, as well as control of other modules, features and devices may be controlled by a set of controls 218, which may be of any type described in previous embodiments. In some embodiments, enclosure 202 is transparent, which may allow a user to see the treatment area.

The medical device 200 includes a fluid inlet 220 near the proximal end 222 of the medical device 200. Fluid inlet 222 may be connected to a suitable gas supply to generate selected species, reactive species and/or plasmas with different temperatures. Some exemplary gases that may be used to generate non-thermal plasma are inert gasses, such as, for example, He, Ar, Ne, Xe and/or the like, combinations thereof, air, or mixtures of inert gases with small percentage (0.5%-20%) of other gases such as air, O₂ and N₂. In addition, mixtures of inert gases with vaporized liquids including water, hydrogen peroxide, ethanol, isopropyl alcohol, n-butanol, with or without additives and the like may be used. Fluids such as saline, that are commonly used for localized cleaning of the treated area can also be introduced through inlet 222. Also, drug molecules dissolved in a proper liquid medium can be delivered through inlet 222. One or more apertures, valves or buttons may control the flow of fluid through the fluid inlet and may be controlled by controls 218. In some embodiments, the gas or vaporized liquid includes drugs, vaccines or the like.

In some embodiments the medical device 200 includes vacuum suction for drawing fluid emitted during plasma generation. Suction helps prevent gases and other fluids from escaping into the body during plasma generation and, when further surrounded by enclosure 202, greatly decreases the risk of escaping fluids. Suction may be achieved by a vacuum pump or the like connected to a suction outlet 224 near the proximal end 222 of the medical device 200. One or more apertures, valves or buttons may control suction through the suction outlet and may be controlled by controls 218. In some embodiments, suction creates slightly negative pressure in deployed enclosure 202. Negative pressure further facilitates uniform plasma generation.

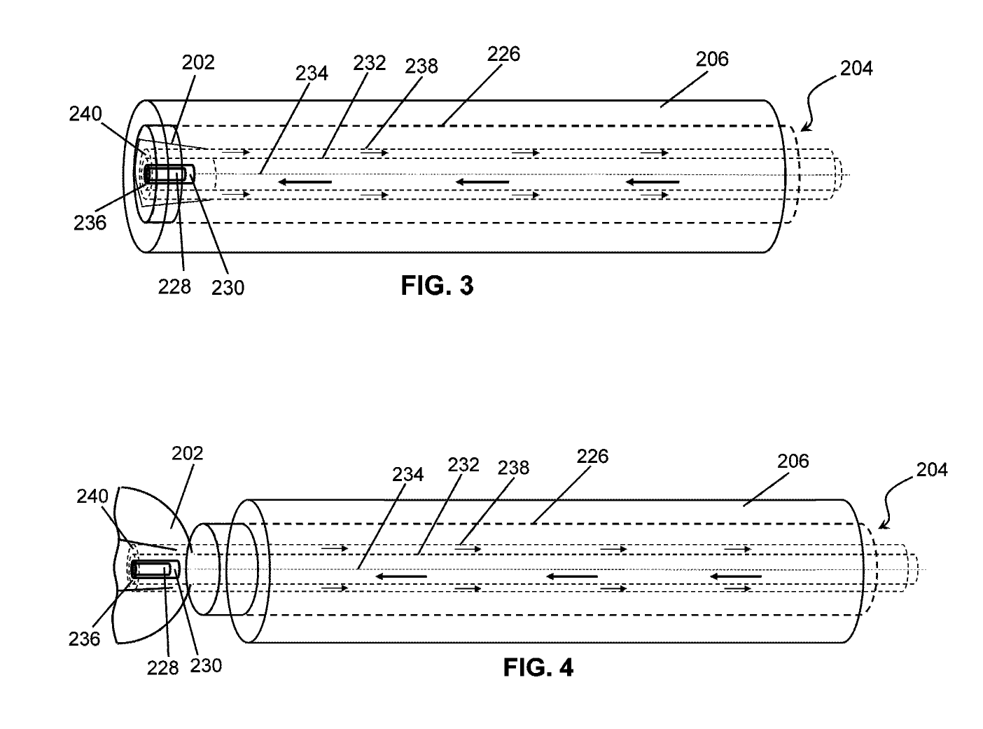

FIGS. 3 and 4 illustrate an embodiment of exemplary plasma generation module 204, disposed in channel 206, with the deployable enclosure 202 undeployed and deployed respectively. Exemplary plasma generation module 204 includes hollow tube 226. The tube 226 may be movable within the channel 206 and may be extended out of the distal end of the medical channel 206.

A high-voltage electrode 228 is located within the tube 226 at the distal end of the tube 226. As described for previous embodiments, the high-voltage electrode 228 may be any suitable shape, form or material. In some embodiments a dielectric barrier 230 surrounds the electrode 228 within the tube 226. In some embodiments a second internal tube 232 within tube 226 serves as the dielectric barrier. The high-voltage electrode 228 is connected or connectable by, for example, wire 234, to a high-voltage source.

Gas may flow from a fluid inlet connected or connectable to the plasma generation module 204 near the proximal end of the module 204. In some embodiments the gas flows through an internal tube 232 within tube 226 and out of a gas outlet 236 at the end of the tube 232. The enclosure 202, which surrounds the outer tube 238, also surrounds the gas outlet 236. Accordingly, a desired gas/vapor may be forced between the insulated electrode 228 and an application surface, such as tissue, to create different reactive species for different types of treatment.

In some embodiments electrode 228 with dielectric barrier 230 is disposed in the same internal tube 232 through which gas flows to the gas outlet 236. In some embodiments the electrode 228 and its power connection may be in one tube and the gas flow in a second tube, both tubes are disposed within larger tube 226. In some embodiments the internal tube 232 may be separately extendable out from the distal end of the larger tube 226, thus allowing the tube 232 to extend into the area surrounded by the enclosure 202 and closer to the area where plasma is to be applied.

In some embodiments vacuum suction is effectuated by air drawn from a suction outlet connected or connectable to the plasma generation module 204 near the proximal end of the module. In some embodiments the air, gas, vapor, mist or the like are drawn through internal suction tube 238 within tube 226, and drawn from a suction inlet 240 at the distal end of the suction tube 238. The enclosure 202, which surrounds the larger tube 238, also surrounds the suction inlet 240.

In some embodiments suction tube 238 may surround and enclose internal tube 232 which houses electrode 228 and through which fluid flows. In some embodiments the electrode 228 and its power connection are in one tube and the fluid flows in a second different tube, both tubes may be disposed within larger suction tube 238. In some embodiments one or more dedicated suction tubes may be disposed separately within the larger tube 226, each being connected to the suction outlet and having its own suction inlet. In some embodiments the suction tube 238 may be separately extendable out from the distal end of the larger tube 226, thus allowing the suction tube 238 to extend into the area surrounded by the enclosure 202 and closer to the area where plasma is to be applied. In some embodiments, enclosure 202 is part of suction tube 238.

In some embodiments the deployable enclosure 202 is secured or fastened to or around the hollow tube 226. When the hollow tube 226 is extended out from the distal end of the medical device 200, the enclosure 202 may be deployed to surround the protruding portion of the tube 226 and to create a barrier around the area where plasma is to be applied. In some embodiments, the enclosure 202, when deployed, has an umbrella or tent shape. In some embodiments the enclosure 202 is made from a flexible material such as rubber, silicone or urethane.

In some embodiments the enclosure 202 includes a support frame or wire made from similar materials or more rigid materials like stainless steel. In some embodiments the enclosure 202 includes a spring mechanism that pushes the enclosure open when it is extended out from the distal end 204 of the medical device 200 and allows compression of the enclosure 202 when it is retracted into the channel 206 of the medical device 200. In some embodiments opening and closing the enclosure is controlled by a wire or other suitable mechanism connected to a set of controls.

Enclosure 202 creates a microenvironment to apply plasma treatment. Various types and combinations of gases, vapors, mists or the like may be injected into the microenvironment to treat an area without the gas, vapor, mist or the like flowing freely into a body cavity.

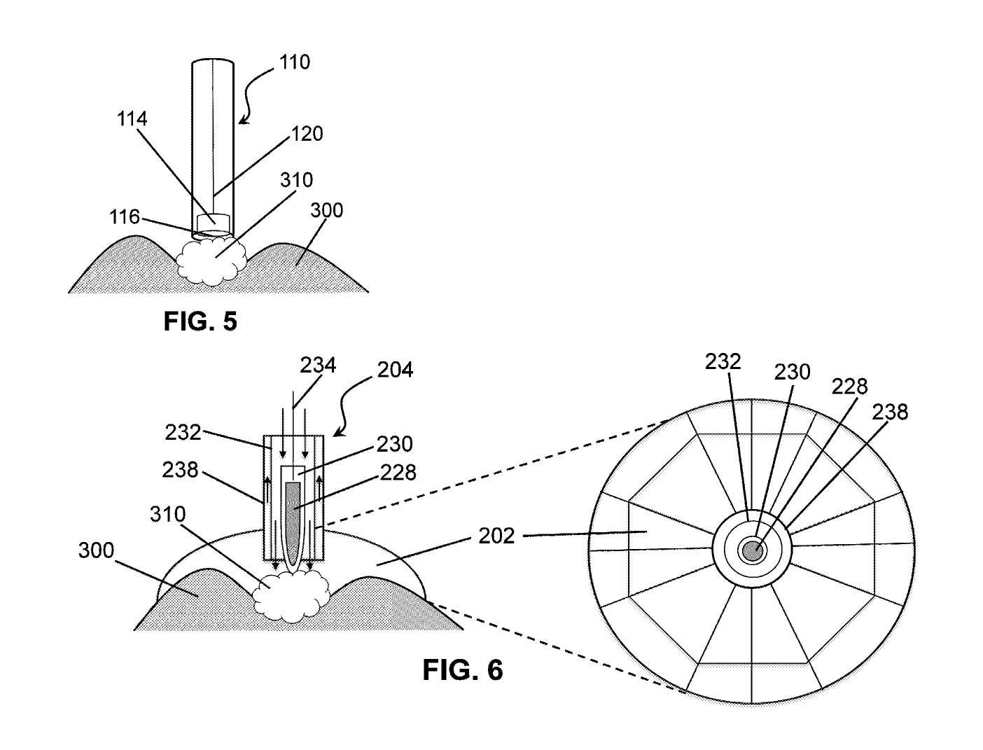

FIG. 5 illustrates exemplary plasma generation module 110 during operation. The plasma generation module 110 may be extended out from a medical device (not shown) and brought into close proximity with an application surface such as tissue 300. The plasma generation module 110 includes a hollow tube closed at its distal end. The tube is made from a suitably dielectric material and serves as the dielectric barrier 116. Cylindrical high-voltage electrode 114 is housed within plasma generation module 110 and is connected to a high-voltage power source (not shown) by conductive wire 120. A high voltage is placed on high-voltage electrode 114 and the tissue 300 is grounded or is under a floating ground potential.

Plasma 310 is formed in the atmosphere between the tissue 300 and dielectric barrier 116 by the application of the high voltage and is used to treat the desired area. The treatment time may be fractions of a second, a few seconds, a few minutes or for longer periods of time. An operator of the medical device need not worry about harmful gases being released into the body during extended use, as no external gas flow is required for plasma generation with the module 110. In some embodiments plasma application is intermittent and mixed with the use of other modules of the medical device. For example the plasma generation module 110 may be used in conjunction with a module for applying medication to a treatment area, such as an aspirator module. An operator of the medical device may alternate between plasma generation for cellular poration to increase the efficacy of absorption, and application of medicine with the aspirator. In another example, a technician may use a pair of forceps to retrieve a tissue sample preceded and/or followed by plasma generation to treat the sampled area.

FIG. 6 illustrates exemplary plasma generation module 204 during operation. The plasma generation module 204 may be extended out from a medical device (not shown) and brought into close proximity with an application surface such as tissue 300. The plasma generation module 204 is a hollow tube and open at its distal end. High-voltage electrode 228 is surrounded by dielectric barrier 230 and housed within internal tube 232. The electrode 228 is connected to a high-voltage power source (not shown) by wire 234. Fluid flows from a fluid inlet (not shown) through the internal tube 232 and out past the electrode 228 at the distal end of the module 204, as indicated by the arrows. Air, gas, vapor, mist or the like is drawn up through the suction tube 238, as indicated by the arrows, and to a suction inlet (not shown). Umbrella-shaped enclosure 202 is deployed and surrounds the distal end of the plasma generation module 204. A high voltage is placed on high-voltage electrode 228 and the tissue 300 is grounded or is a floating ground potential.

Plasma 310 is formed in the fluid by the application of the high voltage as the fluid passes the electrode and is used to treat the desired body part. The treatment time may be for fractions of a second, a few seconds, a few minutes or for longer periods of time. Fluid that flows out from distal end of the plasma generation module 204 is contained within the enclosure 202 and is suctioned back out from the enclosure via suction tube 238. As in previous embodiments plasma application may be intermittent and mixed with the use of other modules of the medical device. Fluid flow and suction may be intermittent as well, for example by allowing fluid flow for plasma generation for a period of time followed by a period of suction before returning to plasma application. Fluid flow and plasma generation may also be simultaneous with suction. Although the embodiments shown and discussed herein are DBD electrodes, the embodiments with an enclosure may use other plasma generators such as, for example, a plasma jet, a corona discharge or the like.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. Therefore, the invention, in its broader aspects, is not limited to the specific details, the representative apparatus and illustrative examples shown and described. Accordingly, departures may be made from such details without departing from the spirit or scope of the applicant's general inventive concept.

1. A medical device for application of DBD plasma comprising:
a proximal end and a distal end;
a first channel extending between the proximal and distal ends and having an opening at the distal end;
a plasma generation module disposed in the first channel, the module having
an electrode,
a dielectric barrier at least partially surrounding the electrode;
wherein when a high voltage source is applied to the electrode, plasma is produced using ambient gas residing between the dielectric barrier and a surface; and
a deployable flexible enclosure.

2. The medical device of claim 1 wherein the deployable flexible enclosure is configured to encloses a treatment area.

3. The medical device of claim 1 wherein the deployed enclosure has an umbrella shape.

4. The medical device of claim 1 wherein the deployable enclosure comprises at least one of rubber, silicone, and urethane.

5. The medical device of claim 1 further comprising a fluid inlet and fluid outlet.

6. The medical device of claim 5 wherein the deployed enclosure surrounds the fluid outlet.

7. The medical device of claim 6 further comprising at least one vacuum suction tube for drawing fluid from the enclosed area.

8. The medical device of claim 1 wherein the device is a flexible endoscope.

9. The medical device of claim 1 wherein the device is a laparoscope.

10. A medical device for application of plasma comprising:
a proximal end and a distal end;
a first channel extending between the proximal end and the distal end and having an opening at the distal end;
a plasma generation module having an electrode,
a fluid inlet at the proximal end of the medical device,
a fluid outlet at the distal end of the medical device, and
a deployable enclosure at the distal end of the medical device wherein the deployable and retractable enclosure surrounds the fluid outlet when deployed;
wherein when a high voltage source is applied to the electrode, plasma is produced.

11. The medical device of claim 12 further comprising at least one vacuum suction tube for drawing fluid out of the deployable enclosure.

12. The medical device of claim 12 wherein the electrode has a needle shape.

13. The medical device of claim 12 further comprising a dielectric barrier.

14. The medical device of claim 12 wherein the plasma generation module is extendable out from the distal end of the medical device so that a portion of the module protrudes from the medical device.

15. The medical device of claim 12 further comprising a high voltage source in circuit communication with the electrode for providing high voltage pulses to the electrode to generate cold plasma.

16. The medical device of claim 17 further comprising a battery for providing a high voltage.

17. The medical device of claim 12 wherein the device is an endoscope.

18. The medical device of claim 12 wherein the device is a laparoscope.

19. A medical device for application of plasma comprising:
a proximal end and a distal end;
a first channel extending between the proximal end and the distal end and having an opening at the distal end;
a plasma generator, and
a deployable enclosure at the distal end that encloses a treatment area and plasma generated by the plasma generator when deployed.

20. A medical device comprising:
a proximal end and a distal end;
a first channel extending between the proximal end and the distal end and having an opening at the distal end;
a treatment device, and
a deployable flexible enclosure at the distal end that encloses a treatment area and the output of the treatment device.
